(19) United States
(12) Patent Application Publication
(10) Pub. No.: US 2010/0324691 A1
(43) Pub. Date: Dec. 23, 2010

(54) GLENOID COMPONENTS AND ASSOCIATED METHODS

(76) Inventor: Yann Brunnarius, Chatuzange Le Goubet (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/817,878

(22) Filed: Jun. 17, 2010

Related U.S. Application Data
(60) Provisional application No. 61/219,021, filed on Jun. 22, 2009.

(30) Foreign Application Priority Data
Jun. 17, 2009 (FR) 0954078

(51) Int. Cl. A61F 2/40

(52) U.S. Cl. 623/19.11

(57) ABSTRACT

A glenoid prosthetic component includes a bearing body for a prosthetic or bony humeral head. The bearing body includes a main portion for articulation with the humeral head and a peripheral portion extending about the main portion. A connection portion resides between the main portion and the peripheral portion and is mechanically weakened in order, in service, to elastically deform relative to the rest of the bearing body under the action of the humeral head.

### CROSS-REFERENCE TO RELATED APPLICATION

The present application claims the benefit of French Patent Application No. FR0954078, filed on Jun. 17, 2009 and also claims the benefit of U.S. Provisional Patent Application No. 61/219,021, filed on Jun. 22, 2009 and entitled Glenoid Implant with a Mechanical Labrum, both of which are incorporated herein by reference in their entireties for all purposes.

### TECHNICAL FIELD

The present invention relates to a glenoid prosthetic component and a set of such components. The invention relates more particularly to the anatomical articular restoration of the socket of the scapula of a human being, by a prosthetic component defining a concave surface on which a prosthetic or natural humeral head rests and articulates.

### BACKGROUND

A large number of current glenoid prosthetic components are satisfactory in their kinematic behavior for shoulder articulation. However, it is found that in service these glenoid components very frequently induce excess stress on the muscle and ligament environment of the shoulder, are rapidly subjected to wear and separations, and/or render shoulder articulation unstable. More generally, it can be said that the kinematic performance of the known glenoid components are reached when the humeral head slides, turns and remains stable on the glenoid component. Moreover, this performance is diminished when at least one of the kinematic parameters (e.g., rotation, sliding, stability) is degraded.

EP-A-1 776 936 proposes coating a rigid prosthetic body with a flexible bearing surface for the humeral head. This flexible bearing surface is made of an elastic material, such as elastomeric polyurethane, that can deform in thickness in order to simulate an anatomical marginal cushion, that cushion sometimes being called by its Latin name, “labrum.” In a general sense, this deformable material induces an interaction with the humeral head that is closer to natural anatomical behavior of the shoulder. However, the aforementioned elastic material used to produce the flexible bearing body does not make it possible, in service, to ensure either satisfactory kinematic performance for the articulation of the shoulder or sufficient resistance to wear.

### SUMMARY

Some inventive aspects relate to a glenoid prosthetic component which, when interacting with a humeral head, combines good kinematic performance while mimicking natural anatomical behavior of the shoulder. In some embodiments, the glenoid prosthetic component includes a bearing body for a prosthetic or bony humeral head, the bearing body including a main articulation portion for interacting with the humeral head, a peripheral portion, and a connection portion between the main portion and the peripheral portion that is mechanically weakened to make the connection portion elastically deformable under action of the humeral head.

In some embodiments, an anatomical glenoid marginal labrum is simulated through use of a peripheral portion of a bearing body, the peripheral portion being produced of a material that has substantially the same rigidity as that used to produce a main portion of the bearing body. If desired, the main and peripheral portions are formed of the same material. For example, by forming the main and peripheral portions of substantially the same, general material, the main portion and the peripheral portion are more readily formed as a single piece which allows more efficient manufacturing processes. Use of the same material for the main and peripheral portions is facilitated through use of the connection portion, which is a mechanically weakened zone between the main portion and the peripheral portion. The connection portion is elastically deformable relative to the rest of the bearing body and is designed so that, under the action of the humeral head on the peripheral portion, the connection portion elastically deforms. In some embodiments, the deformation of the connection portion results in the peripheral portion exhibiting a behavior that is more similar to that of a glenoid anatomical marginal labrum. For example, the peripheral portion helps restrain, retain, and recenter the humeral head when in service by moving away from the central region of the main portion of the bearing body. The main portion optionally has a geometry and a hardness that helps ensure appropriate, lasting kinematic performance to support articulation between the bearing body and the humeral head.

Some embodiments relate to a glenoid prosthetic component including a bearing body for a prosthetic or natural humeral head. The bearing body has a first face for bearing against the humeral head and a second face opposite the first face. The bearing body includes a main portion for articulation with the humeral head, a peripheral portion surrounding the main portion, and a connection portion between the main portion and the peripheral portion. The connection portion is mechanically weakened such that the connection portion is elastically deformable relative to the rest of the bearing body under loading by the humeral head.

Some other embodiments relate to a kit of parts for a shoulder prosthesis, the kit including at least two glenoid prosthetic components. Each glenoid component includes a bearing body for a prosthetic or natural humeral head, the bearing body having a first face for bearing against the humeral head and a second face opposite the first face. The bearing body includes a main portion for articulation with the humeral head, a peripheral portion surrounding the main portion, and a connection portion between the main portion and the peripheral portion. The connection portion is mechanically weakened such that the connection portion is elastically deformable relative to the rest of the bearing body under loading by the humeral head. In at least one direction passing through two portions of the connection portions of the glenoid prosthetic components that are diametrically opposite to the central regions of the main portions of the glenoid prosthetic components, the bearing bodies of the glenoid prosthetic components have different dimensions.

Still other embodiments relate to a method of implanting a shoulder prosthesis. The method includes securing a bearing body of a glenoid prosthetic component to a glenoid socket, the bearing body having a first face for bearing against the humeral head and a second face opposite the first face that is secured against the glenoid socket. A peripheral portion of the bearing body is loaded with a humeral head to elastically deform a connection portion of the bearing body that resides between the peripheral portion and a main portion of the bearing body. The connection portion is mechanically weakened relative to the rest of the bearing body. The peripheral portion is unloaded such that the connection portion elastically recovers.

While multiple embodiments are disclosed, still other embodiments of the present invention will become apparent to those skilled in the art from the following detailed description, which shows and describes illustrative embodiments of the invention. Accordingly, this summary, the drawings, and detailed description are to be regarded as illustrative in nature and not restrictive.

### BRIEF DESCRIPTION OF THE DRAWINGS

FIG. 1 is a schematic diagram of a shoulder prosthesis implanted in a patient, according to some embodiments.

FIG. 2 is a view in perspective showing a glenoid component of the shoulder prosthesis of FIG. 1, according to some embodiments.

FIG. 3 is an elevation view in the direction of arrow III shown in FIG. 2, according to some embodiments.

FIGS. 4 and 5 are sectional views on lines IV-IV and V-V of FIG. 3, respectively, according to some embodiments.

FIG. 6 is an enlarged view of the circled detail VI of FIG. 4, according to some embodiments.

FIG. 7 is a schematic view of the glenoid component of FIGS. 1 to 6 and two other glenoid components, the three glenoid components belonging to a kit or set of parts, according to some embodiments.

FIGS. 8 to 12 illustrate another glenoid component, according to some embodiments.

While the invention is amenable to various modifications and alternative forms, specific embodiments have been shown by way of example in the drawings. The intention, however, is not to limit the invention to the particular embodiments depicted. On the contrary, the invention is intended to cover all modifications, equivalents, and alternatives falling within the scope of the invention as defined by the appended claims.

### DETAILED DESCRIPTION

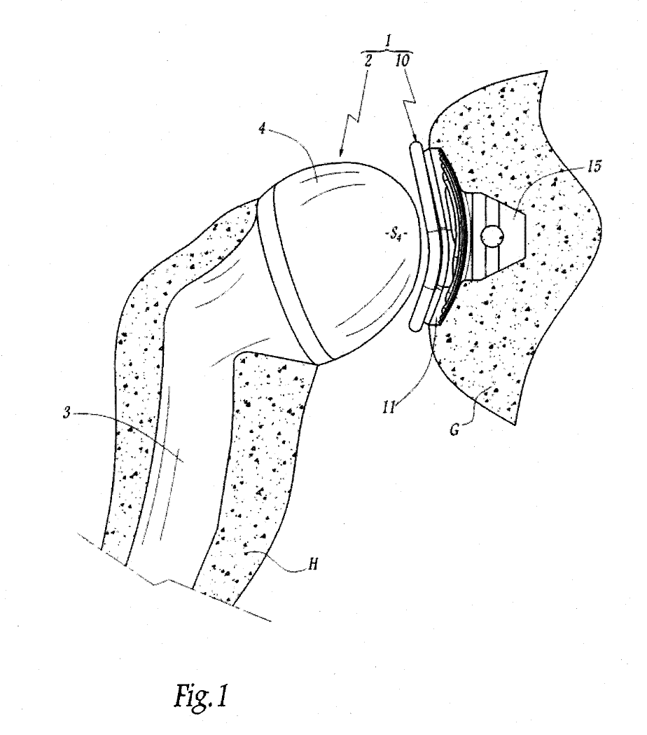

FIG. 1 shows a total shoulder prosthesis 1 comprising a glenoid component 10 fixed to a glenoid socket G of the scapula of a patient and a humeral component 2 fixed to the humerus H of the patient. The glenoid component 10 and humeral component 2 are generally adapted for articulating contact with one another. As shown, the humeral component 2 comprises a shank 3, adapted to be fixedly anchored in the medullary cavity of the humerus H, and a humeral head 4, fixedly supported by the shank 3 and externally delimiting a convex surface S₄. As shown, the convex surface S₄ is of a generally hemispherical shape, although other configurations are contemplated.

When the prosthesis 1 is in service, the convex surface S₄ of the humeral head 4 rests and articulates against the glenoid component 10, as explained in detail below. In other embodiments, rather than the glenoid component 10 interacting with a prosthetic humeral head, such as the head 4, the bony head of the humerus H directly contacts and articulates with the glenoid component 10. Where the glenoid component directly articulates with the humerus, the shoulder prosthesis 1 is optionally characterized as a partial prosthesis.

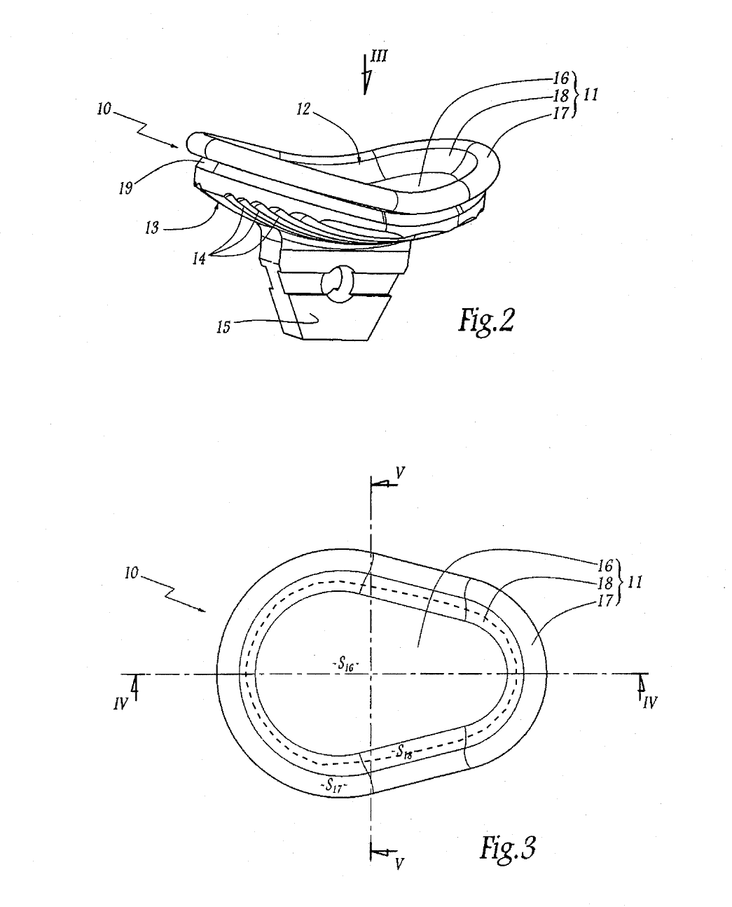

As shown in greater detail in FIGS. 2 to 6, the glenoid component 10 includes a one-piece body 11, also described as a unitary body, that is generally cup-shaped. In some embodiments, the body 11 has two main faces that oppose one another—a first face 12 that is essentially hollow and adapted to be turned toward the humeral head 4 and a second face 13 that is generally domed or flat and adapted to be pressed and secured against the bone of the glenoid socket G.

As shown, the second face 13 includes furrows 14, also described as scallops, adapted to directly engage the bony surface of the glenoid socket G. The second face 13 also forms a protruding keel 15, designed to be anchored in the bone of the socket G. As shown, the keel 15 has a hole into which cement is able to be injected in order to lock and stabilize the second face 13 relative to the glenoid socket G. The keel 15 is additionally or alternatively anchored to the glenoid socket G by a transverse screw as desired. In other embodiments, the second face 13 includes several protruding studs that are anchored into the bony material of the glenoid socket G, one or more through-holes in which screws are received, other members for bony anchoring with the glenoid socket G, and/or a hydroxyapatite-based coating to make osteo-integration easier, as well as other additional or alternative fixation means.

As shown in FIGS. 3 to 5, the body 11 includes a main portion 16, also described as a main body, which, on the side of the second face 13, supports the furrows 14 and the keel 15, a peripheral portion 17, also described as a peripheral edge, which extends around the main portion 16, and a connection portion 18, also described as a connection zone, connecting together the main portion 16 and the peripheral portion 17.

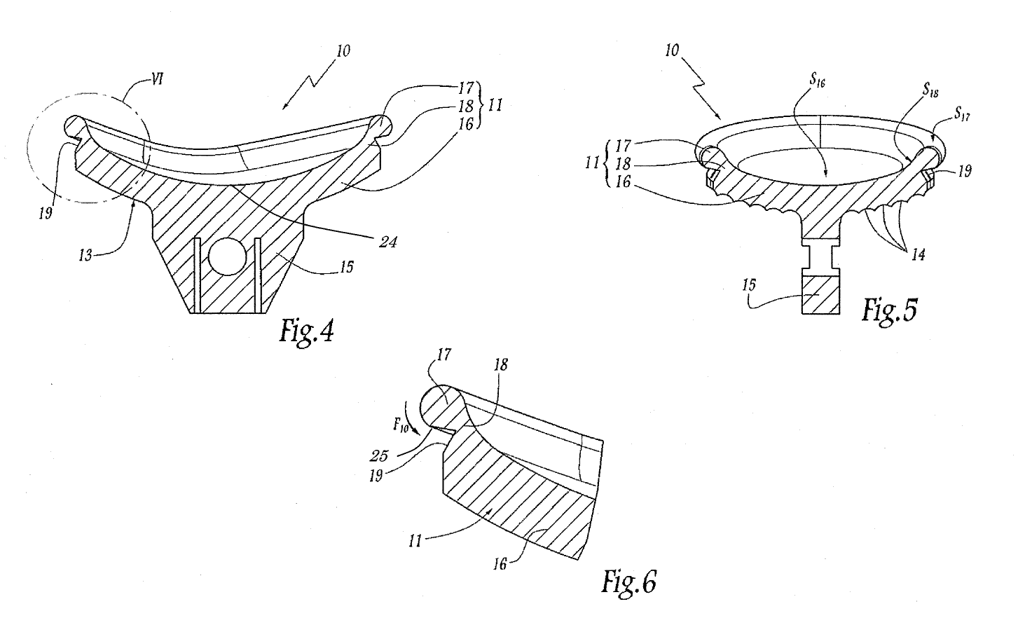

The peripheral portion 17 has, on the first face 12 of the body 11, a convex surface S₁₇ which, in cross-section (FIGS. 4 and 5), has a curved profile. As shown, the curved profile corresponds substantially to an arc of a circle. The peripheral portion 17 is optionally referred to as a rim, a lip, or an edge as appropriate. As shown, the peripheral portion 17 is substantially thinner than the main portion 16, although in other embodiments the peripheral portion 17 is substantially the same thickness, or of a greater thickness, than the main portion 16.

The main portion 16 and the connection portion 18 have, on the first face 12 of the body 11, concave surfaces S₁₆, S₁₈, respectively. The concave surface S₁₆ is adapted to interact in an articular manner with the convex surface S₄ of the humeral head 4 (e.g., in both rotation and sliding), where the respective geometries of the surfaces S₄, S₁₆ are configured to interact to reproduce anatomical articular behaviors of the shoulder. As understood with reference to the sectional views of FIGS. 4 and 5, in some embodiments, a curvature of the surface S₁₈ is larger than that of the surface S₁₆.

In some embodiments, when observing the body 11 in a direction at right angles to the first face 12, as in FIG. 3, the respective peripheral contours of the main portion 16, connection portion 18, and peripheral portion 17 have shapes similar to a contour of the glenoid socket G. For example, the contours of the portions 16, 17, and 18 are optionally generally oval-shaped or pear-shaped, where a pear-shaped profile is one that is elongate at the rounded opposite ends, with one of these ends having a radius of curvature that is greater than the other end. In some implementations, the pear-shape more faithfully reproduces the corresponding anatomy of the glenoid socket G or is otherwise advantageous.

As shown in FIG. 6, the connection portion 18 is furnished with a peripheral groove 19 formed in the second face 13 of the body 11. In some embodiments, the groove 19 forms a hollow in the material forming the connection portion 18, reducing the thickness of the connection portion 18 relative to the thickness of the main portion 16 and the thickness of the peripheral portion 17. As shown, the groove 19 extends continuously about the entire periphery of the body 11 without interruption. In other embodiments, the groove 19 is interrupted, also described as discontinuous or segmented, and extends about multiple portions of the periphery of the body 11. For example, the broken line in FIG. 6 schematically represents a discontinuous, segmented groove 19 extending about the main portion 16, according to some other embodiments.

As shown, the groove 19 has a V-shaped cross-section, although other shapes are contemplated, including U-shaped profiles, W-shaped profiles, accordion-like profiles, and others. Moreover, although a single groove is shown, multiple spaced apart connection portions are contemplated, including a plurality of concentric contour lines, for example. In use, the groove 19 mechanically weakens the connection portion 18 to provide for elastic deformation of the body 11 at the connection portion 18 for relative movement between the main portion 16 and the peripheral portion 17.

In some embodiments, when the prosthesis 1 is implanted and the patient articulates his or her shoulder, the humeral head 4 rests against the body 11 on the first face 12. As the humeral head 4 is interacting centrally with the main portion 16, the surfaces S₄ and S₁₆ interact in an articulated manner as explained above. In certain stress configurations, or loading conditions, the humeral head 4 moves away from the central region 24 of the main portion 16 and, thus moves closer to the connection portion 18 and the peripheral portion 17.

In some embodiments, when the convex surface S₄ of the humeral head 4 rests on the surface S₁₈ of the connection portion 18, the more curved concavity of the surface S₁₈ relative to the surface S₁₆ tends to bring the humeral head 4 toward the central region 24 of the main portion 16. In turn, when the humeral head 4 rests against the peripheral portion 17, the convex surface S₁₇ of the peripheral portion 17 simulates the presence of an anatomical glenoid marginal labrum, tending to hold the humeral head 4 by abutment. The resting of the humeral head 4 against the peripheral portion 17 generates a tilting torque on the peripheral portion 17 relative to the main portion 16, the tilting torque tending to bring the edges 25 of the groove 19 closer together as indicated by the arrow F₁₀ in FIG. 6.

In some embodiments, flexing of the groove 19 results in elastic deformation of the connection portion 18 such that, when the pressure of the humeral head 4 relaxes, the energy of deformation accumulated in the connection portion 18 is released elastically to recover its unloaded or less-loaded configuration. Upon release of the pressure from the humeral head 4, the edges 25 of the groove 19 separate in order to resume their initial configuration so that the peripheral portion 17 pushes the humeral head 4 toward the central region 24 of the main portion 16.

Thus, in some embodiments, the groove 19 confers on the peripheral portion 17 capacity for elastic retention of the humeral head 4 while also facilitating forming the peripheral portion 17 of the same, relatively hard material as that forming the main portion 16. In particular, in some embodiments, the main portion 16 is hard, or is made of a material that resists deformation and wear under the action of the stresses, or loads, routinely encountered during shoulder articulation, which helps ensure accurate and lasting articular mechanical interaction between the main portion 16 and the humeral head 4. In some embodiments, the material used to produce the main portion 16 and, more generally, to produce the whole body 11, including the peripheral portion 17 and the connection portion 18, is high-density polyethylene, a metal alloy, pyrolytic carbon, polyether ether ketone (PEEK), combinations thereof, and others.

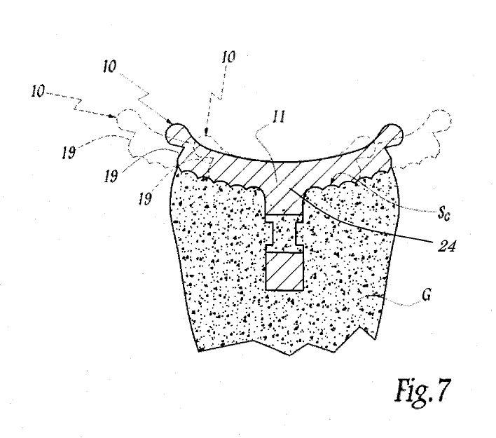

In some embodiments, the glenoid component 10 is available to the surgeon in a plurality of different sizes during prosthesis implantation. For example, the surgeon is optionally provided with three differently sized glenoid components 10 as shown in FIG. 7. As shown, in addition to the glenoid component 10 considered in the previous figures, which is represented in solid lines, two other glenoid components 10, respectively smaller and larger than the component 10 of the previous figures, are shown in dashed lines. In some embodiments, the three glenoid components 10 shown in FIG. 7 have similar bodies 11, except for the arrangements of their second face 13, respectively, making it possible to fix the glenoid components 10 to the glenoid socket G. In particular, the three glenoid components 10 have different respective values for their dimensions in any direction passing through two portions of their grooves 19, respectively, that are diametrically opposed about their central regions 24.

In some embodiments, by providing multiple glenoid components 10, the surgeon is able to select a position of the groove 19 relative to the glenoid socket G of the patient being operated on. For example, as shown schematically in FIG. 7, the smallest implant of the three available components 10 results in positioning the groove 19 inside the bony surface S_{G} of the glenoid socket G, while the largest implant of the three components 10 results in positioning the groove 19 outside the bony surface S_{G}. In an intermediate manner, the implant of the last of the three available components results in positioning the groove 19 substantially in line with the peripheral surround of the bony surface S_{G}. Though three examples for relative positions of the groove 19 have been described, other positions and/or combinations of the above referenced positions are contemplated.

In some embodiments, the mechanical behavior of the connection portion 18 is influenced by the relative positioning of the groove 19 and the bony surface S_{G} of the glenoid socket G. For example, depending on whether the groove 19 is on the outside of, on the inside of, or even with the periphery of the bony surface S_{G}, the edges 25 of the groove 19 separate from one another with more or less ease, that is to say under the action of a greater or lesser force applied to the peripheral portion 17 by the humeral head 4. Therefore, particularly in pathological cases of reconstruction of the shoulder articulation, the surgeon can choose the size of the glenoid component 10 that he considers to be the most appropriate.

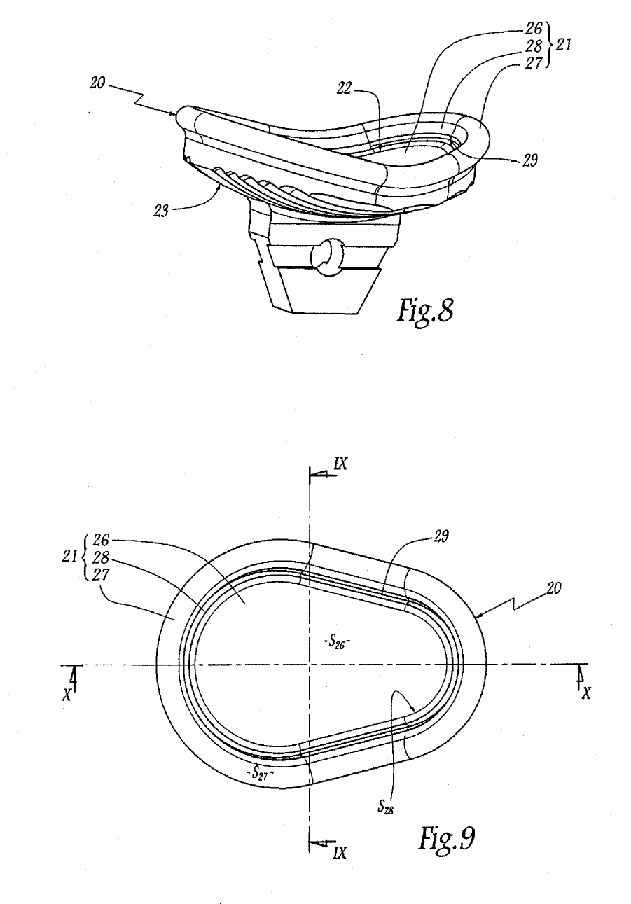

FIGS. 8 to 12 show another glenoid component 20, which includes some features that are substantially similar to those of the glenoid component 10, the glenoid component 20 including a bearing body 21 for the humeral head 4. The body 21 includes a main portion 26 of articulation with the head 4, a peripheral portion 27 simulating an anatomical glenoid marginal labrum, and a connection portion 28 between the main portion 26 and the peripheral portion 27. On a first face 22 of the body 21, turned toward the humeral head 4 in service, the main portion 26, the peripheral portion 27, and the connection portion 28 have surfaces S₂₆, S₂₇ and S₂₈, that are respectively concave, convex, and concave, and function similarly to the surfaces S₁₆, S₁₇ and S₁₈ of the component 10. A second face 23 of the body 21 resides opposite the first face 22, is like the second face 13 of the body 11, and is suitable to be pressed against and secured to the bone of the glenoid socket G.

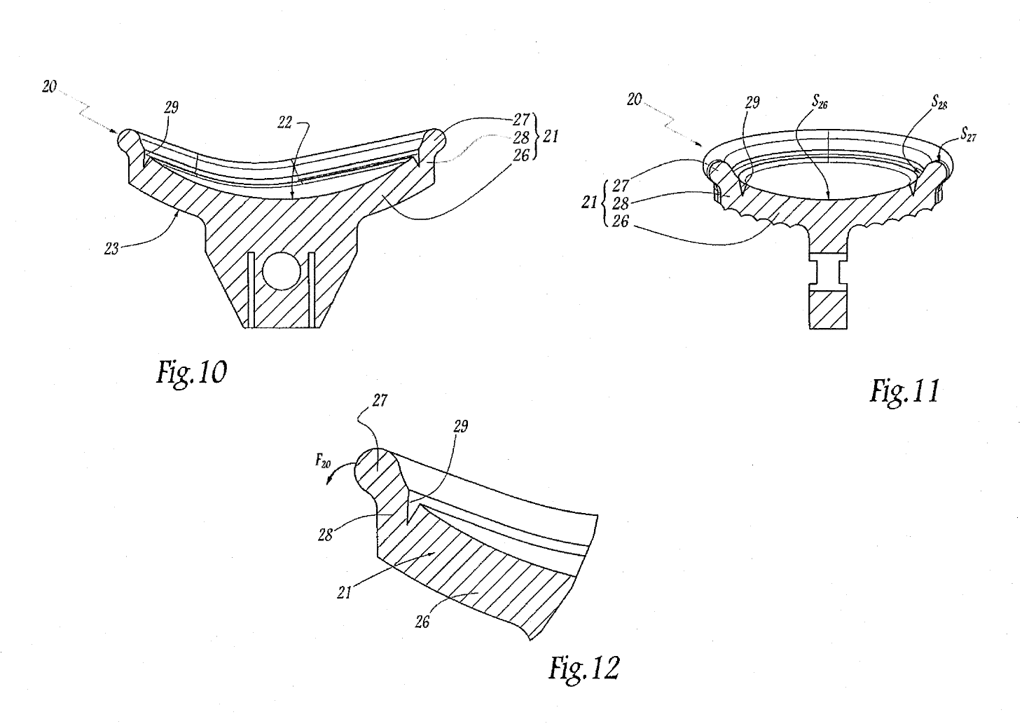

As is clearly visible in FIGS. 10 to 12, the component 20 differs from the component 10 by the arrangement of the connection portion 28, which is designed to mechanically weaken the body 21 to provide flexibility, or the ability to more readily deform. Specifically, in the glenoid component 20, the groove 19 of the glenoid component 10 is replaced with a furrow 29 delimited in the first face 22 of the body 21.

In service, the furrow 29 allows, in a similar manner as the groove 19, elastic deformation of the connection portion 28 when the humeral head 4 is pressed on the peripheral portion 27, with the edges of the furrow 29 moving away from and closer to one another. More precisely, as indicated by the arrow F₂₀ in FIG. 12, the bearing action of the humeral head 4 tends to separate the edges of the furrow 29; then, by elastic return of the material forming the connection portion 28, the aforementioned edges tend to move closer to one another.

Various modifications and additions can be made to the exemplary embodiments discussed without departing from the scope of the present invention. For example, the connection portion 18 optionally includes several grooves 19 placed one after the other in a direction connecting the main portion 16 and the peripheral portion 17 and/or the connection portion 28 optionally includes several furrows 29. Additionally, the features of the components 10 and 20 are optionally augmented or substituted with one another according to various embodiments—for example, a connection portion between a main portion and a peripheral portion is optionally provided with both having at least one groove 19 and at least one furrow 29. In other words, while the embodiments described above refer to particular features, the scope of invention also includes embodiments having different combinations of features and embodiments that do not include all of the above described features.

1. A glenoid prosthetic component comprising a bearing body for a prosthetic or natural humeral head, the bearing body having a first face for bearing against the humeral head and a second face opposite the first face and the bearing body including a main portion for articulation with the humeral head, a peripheral portion surrounding the main portion, and a connection portion between the main portion and the peripheral portion, the connection portion being mechanically weakened such that the connection portion is elastically deformable relative to the rest of the bearing body under loading by the humeral head.

2. The component of claim 1, wherein the connection portion is mechanically weakened along an entire periphery of the bearing body.

3. The component of claim 1, wherein the connection portion is mechanically weakened along a portion of a periphery of the bearing body.

4. The component of claim 1, wherein the connection portion is mechanically weakened along a plurality of segments along a periphery of the bearing body.

5. The component of claim 1, wherein the connection portion is mechanically weakened by at least one groove delimited in the second face of the bearing body.

6. The component of claim 1, wherein the connection portion is mechanically weakened by at least one furrow delimited in the second face of the bearing body.

7. The component of claim 1, wherein the peripheral portion defines a convex surface on the first face of the bearing body and the connection portion defines a concave surface on the first face of the bearing body.

8. The component of claim 7, wherein the main portion defines a concave surface on the first face of the bearing body, the curvature of the concave surface of the main portion being smaller than the concave surface of the connection portion.

9. The component of claim 1, wherein the second face of the bearing body is adapted to bear against and be secured to the bone of a glenoid socket.

10. The component of claim 1, wherein the bearing body is a single, unitary piece.

11. A kit of parts for a shoulder prosthesis, the kit including at least two glenoid prosthetic components, wherein each glenoid component comprises a bearing body for a prosthetic or natural humeral head, the bearing body having a first face for bearing against the humeral head and a second face opposite the first face and the bearing body including a main portion for articulation with the humeral head, a peripheral portion surrounding the main portion, and a connection portion between the main portion and the peripheral portion, the connection portion being mechanically weakened such that the connection portion is elastically deformable relative to the rest of the bearing body under loading by the humeral head, wherein in at least one direction passing through two portions of the connection portions of the glenoid prosthetic components that are diametrically opposite to the central regions of the main portions of the glenoid prosthetic components, the bearing bodies of the glenoid prosthetic components have different dimensions.

12. A method of implanting a shoulder prosthesis, the method comprising:
securing a bearing body of a glenoid prosthetic component to a glenoid socket, the bearing body having a first face for bearing against the humeral head and a second face opposite the first face that is secured against the glenoid socket;
loading a peripheral portion of the bearing body with a humeral head to elastically deform a connection portion of the bearing body that resides between the peripheral portion and a main portion of the bearing body, the connection portion being mechanically weakened relative to the rest of the bearing body; and
unloading the peripheral portion of the bearing body such that the connection portion elastically recovers.

13. A method of implanting a shoulder prosthesis, the method comprising:
securing a bearing body of a glenoid prosthetic component to a glenoid socket, the bearing body having a first face for bearing against the humeral head and a second face opposite the first face secured against the glenoid socket;
loading a peripheral portion of the bearing body with a humeral head to elastically deform a connection portion of the bearing body that resides between the peripheral portion and a main portion of the bearing body, the connection portion being mechanically weakened relative to the rest of the bearing body; and
unloading the peripheral portion of the bearing body such that the connection portion elastically recovers.

14. The method of claim 13, wherein loading the peripheral portion of the bearing body with a humeral head includes loading the peripheral portion of the bearing body with an artificial humeral head.

15. The method of claim 13, wherein loading the peripheral portion of the bearing body with a humeral head includes loading the peripheral portion of the bearing body with a natural humeral head.

16. The method of claim 13, further comprising securing the bearing body of the glenoid prosthetic component to the glenoid cavity such that the connection portion is positioned outside of a periphery of the glenoid cavity.

17. The method of claim 13, further comprising securing the bearing body of the glenoid prosthetic component to the glenoid cavity such that the connection portion is aligned with a periphery of the glenoid cavity.

18. The method of claim 13, further comprising securing the bearing body of the glenoid prosthetic component to the glenoid cavity such that the connection portion is positioned inside of a periphery of the glenoid cavity.

19. The method of claim 13, wherein loading a peripheral portion of the bearing body with a humeral head to elastically deform the connection portion of the bearing body includes causing edges of a groove formed into the second face to move closer to one another.

20. The method of claim 13, wherein loading a peripheral portion of the bearing body with a humeral head to elastically deform the connection portion of the bearing body includes causing edges of a furrow formed into the first face to move closer to one another.
